(19) United States
(12) Patent Application Publication
(10) Pub. No.: US 2018/0130548 A1
(43) Pub. Date: May 10, 2018

(54) Using an NFC Enabled Mobile Device To Manage Digital Medical Artifacts

(71) Applicant: Michelle Fisher, Oakland, CA (US)

(72) Inventor: Michelle Fisher, Oakland, CA (US)

(73) Assignee: Blaze Mobile, Berkeley, CA (US)

(21) Appl. No.: 13/218,879

(22) Filed: Aug. 26, 2011

Related U.S. Application Data
(63) Continuation-in-part of application No. 13/216,098, filed on Aug. 23, 2011, now abandoned.
Application No. 13/216,098 is a continuation-in-part of application No. 13/215,069, filed on Aug. 22, 2011, now abandoned.
Application No. 13/215,069 is a continuation-in-part of application No. 13/213,840, filed on Aug. 19, 2011, now abandoned.
Application No. 13/213,840 is a continuation-in-part of application No. 13/208,247, filed on Aug. 11, 2011, now abandoned.
Application No. 13/208,247 is a continuation-in-part of application No. 13/184,209, filed on Jul. 15, 2011, now abandoned.
(60) Provisional application No. 61/442,384, filed on Feb. 14, 2011.

(51) Int. Cl. G16H 10/60; G06Q 20/32; G06Q 20/10

(52) U.S. Cl. CPC G16H 10/60; G16H 15/00; G06Q 20/105; G06Q 20/32

(57) ABSTRACT

This invention describes how users can use an NFC enabled mobile device to manage medical digital artifacts such as a medical ID, basic patient information, patient contact information, emergency contact information, primary care physician information, health insurance information including co-pay and deductibles, prescriptions, office visit summary, appointment cards, Electronic Medical Records (EMR), lab results, blood type, organ/donor status, vital signs, diagnostic data, immunization records, payments and transaction history, pictures, etc Using an NFC enabled device, users can also pay for prescriptions as well as pay for medical services.

### CLAIM OF PRIORITY

This application is a continuation in part and claims priority to U.S. patent application Ser. No. 13/216,098 entitled “Streamlining NFC Transactions Using A Mobile Communication Device” filed on Aug. 23, 2011 which is a continuation in part and claims priority to U.S. patent application Ser. No. 13/215, 069 entitled “Transferring Data From an NFC Enabled Mobile Device to a Remote Device” filed on Aug. 22, 2011 which is a continuation in part of U.S. patent Ser. No. 13/213,840, entitled “Transferring Data Between NFC Enabled Mobile Devices With Multiple Secure Elements” filed on Aug. 19, 2011 which is a continuation in part and claims priority to U.S. patent Ser. No. 13/208,247, entitled “Transferring Data Between NFC Enabled Mobile Devices ” filed on Aug. 11, 2011 which is a continuation in part of U.S. patent Ser. No. 13/184,209, entitled “Method and Systems of loading and unloading digital artifacts between a mobile device with an associated secure element and other remote devices” filed on Jul. 15, 2011 which claims priority to U.S. Provisional Patent Application No. 61/442,384, entitled “Method and Systems of loading and unloading digital between a mobile device with an associated secure element and other remote devices” filed on Feb. 14, 2011. All of the above-referenced patent applications are incorporated by reference herein in their entirety

### RELATED APPLICATIONS

Other patents referenced include U.S. patent application Ser. No. 11/944,267, entitled “Method and System for Delivering Information to a mobile communication device based on consumer transactions”, filed Nov. 21, 2007 and U.S. patent application Ser. No. 11/956,261 entitled “Method and System for Delivering Customized Information To A Mobile Communication Device Based on User Affiliations”, filed Dec. 13, 2007. All of the above-referenced patent applications are incorporated by reference herein in their entirety

### FIELD OF INVENTION

The present invention relates to data communications and wireless devices.

### BACKGROUND OF THE INVENTION

Mobile communication devices—e.g., cellular phones, personal digital assistants, and the like—are increasingly being used to conduct payment transaction. Such payment transactions can include, for example, purchasing goods and/or services, tickets, bill payments, and transferring funds between bank accounts. Because of bigger screens, better user input with touch screens increased processing capability, and faster more reliable wireless mobile devices, can be used to manage electronic medical records.

### BRIEF SUMMARY OF THE INVENTION

The system is configured with a mobile device (with client application also referred to as a mobile wallet), a secure element that is associated with the mobile device, NFC Point Of Sale System which is capable of processing financial transactions (including 2-way wireless terminal capable of sending and transmitting), NFC Point-Of-Presence Terminal which is capable of processing non-financial transactions (including 2-way wireless terminal capable of sending and transmitting), an NFC Point-of-Entry Device which is capable of providing access to venues including 2-way wireless terminal capable of sending and transmitting, a management server, a Trusted Service Manager (TSM) and several 3rdParty artifact provider/aggregator.

In the delivery scenario, when the user holds an NFC enabled mobile communication device with a secure element coupled to it in proximity of a suitable equipped POS/POE/POP with NFC, the NFC POS/POE/POP device will trigger an application residing in the secure element (which is different than an application residing on the mobile communication device) to power on and activate an application residing in the secure element, transfer data or digital artifacts (e.g debit/prepaid/credit card, receipts, tickets, advertisements, coupons, loyalty points, gift cards, vouchers, content (video, image, ringtone, movie), games, documentation, business cards, tickets, hotel key, health care records, security key, automobile key, etc.) from/to the secure element, and the secure element may issue commands and/or respond to commands just to name a few.

Using this system, at the time of a transaction, a user can use the mobile wallet software (on a mobile computing device) and associated secure element, in conjunctions with the POP/POS/POE to:
Store medical ID
Store patient contact information such as address, phone number, employer contact info
Store emergency contact information
Store basic patient information such as gender, age, date of birth, blood type, race, etc
Store physician names and contact information
Store allergies
Store a list of vaccinations
Store vital signs such as temperature, blood pressure, pulse, respiratory rate, “pain” level, glucose level, cholesterol, height, weight, etc
Store insurance information including list of approved doctors approved labs, approved drugs, approved generic drugs, health care coverage, out of pocket costs, co-pay costs
Payment methods—credit, debit, prepaid, checking account number
Store prescriptions
Store doctors' orders for use of prescriptions
Pay for prescriptions
Store XRays
Store lab results
Pay for lab tests
Store hospital ID bracelet
Store medication used in hospital
Schedule of doctor appointments
Store organ status and donor information
Transfer digital medical artifacts (e.g medical ID, basic patient information, patient contact information, emergency contact information, primary care physician information, health insurance information including co-pay and deductibles, prescriptions, office visit summary, appointment cards, Electronic Medical Records (EMR), lab results, blood type, organ/donor status, vital signs, diagnostic data, immunization records, payments and transaction history, pictures, etc) to remote devices

Some of the challenges that an NFC enabled mobile device can address include, but are not limited to the following use cases:

Elderly/Senior Citizens—They may have a sudden health care need with a sensitive need to have a list of medical information, allergies, and prescriptions readily available for urgent health care. Care givers may not have access to their medical information.

ER Patients Their identity may not be known when they arrive at ER and require emergency medical treatment. So, it is urgent to identify who they are, medical history, allergies, and prescriptions, etc.

New Patients—If a new patient is visiting a new doctor for a diagnosis and potentially time sensitive, treatment, patients will need to have medical information, allergies, and prescriptions readily available which may take weeks to receive from previous physician.

Children—Children may not have an ID with them and be on a field trip, sporting event, camping trip, friend's house, etc. So, if there is a health issue, it will be important to be able to confirm their identity, medical information, allergies, and prescriptions readily available for urgent health care, etc.

Frequent international travelers—Frequent travelers need to track their shots and immunizations for travel abroad. If they may have a sudden health care need, they will need to have access to medical information, allergies, and prescriptions readily available for urgent health care.

Patients with Alzheimer's or other conditions that impact memory. They may get lost and not remember who they are or how to get back home or have any identification with them. So, they health care professionals will need to he able to identify them as well as their medical history, allergies, and prescriptions readily available for urgent health care.

Critically serious patients in ICU or prenatal may have require very specific medications and dosages.

Patients who require daily or constant monitoring for insulin levels, asthma, high blood pressure, heart pace maker, etc.

This invention enables patients and health care professionals (nurse, doctor, pharmacist, etc) to manage digital medical artifacts medical digital artifacts (e.g. medical ID, basic patient information, patient contact information, emergency contact information, primary care physician information, health insurance information including co-pay and deductibles, prescriptions, office visit summary, appointment cards, Electronic Medical Records (EMR), lab results, blood type, organ/donor status, vital signs, diagnostic data, immunization records, payments and transaction history, pictures, etc) using an NFC enabled mobile device.: This is accomplished when the user holds, “waves”, or “taps” their NFC enabled device in proximity to an NFC Point-Of-Presence (POP) device, Point-Of-Entry (POE), Point-Of-Sale (POS) device, smart poster, or another NFC enabled device. Upon doing so, the POP/POE/POS/etc activates the secure element associated with the NFC enabled mobile device and powers the secure element through induction. So, the secure element need not have its own power source nor does the secure element require use of the battery inside of the mobile device. Correspondingly, if the battery in the mobile device is dead, the user may not be able to use the mobile device to place a phone call, receive an SMS, or surf the web, but they will be able to transfer information from their secure element (e.g. medical ID) since the secure element is powered by the remote NFC enabled POP/POS/POE device. The secure element can also receive digital artifacts from a POP/POS/POE device such as Xrays, prescriptions, appointment cards, etc even if the battery inside of the mobile communication device lacks power. This communication is specified, for example, in the ISO 14443A/B standard and ISO 15693 just to name a few.

### BRIEF DESCRIPTION OF THE DRAWINGS

FIG. 1 illustrates a block diagram of a communication system including a wireless mobile communication device and a management server in accordance with one implementation.

FIG. 2 illustrates a block diagram of a mobile communication device in accordance with one implementation.

FIG. 3 illustrates a block diagram of a secure element in accordance with one implementation.

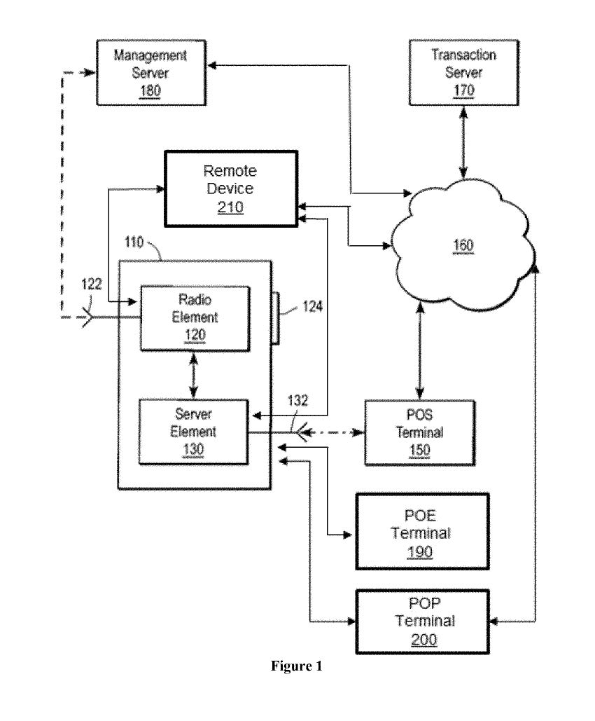

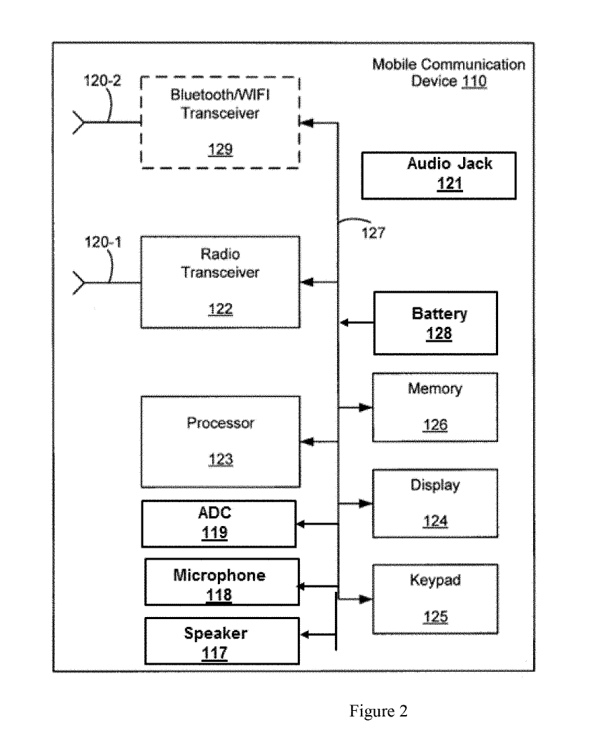

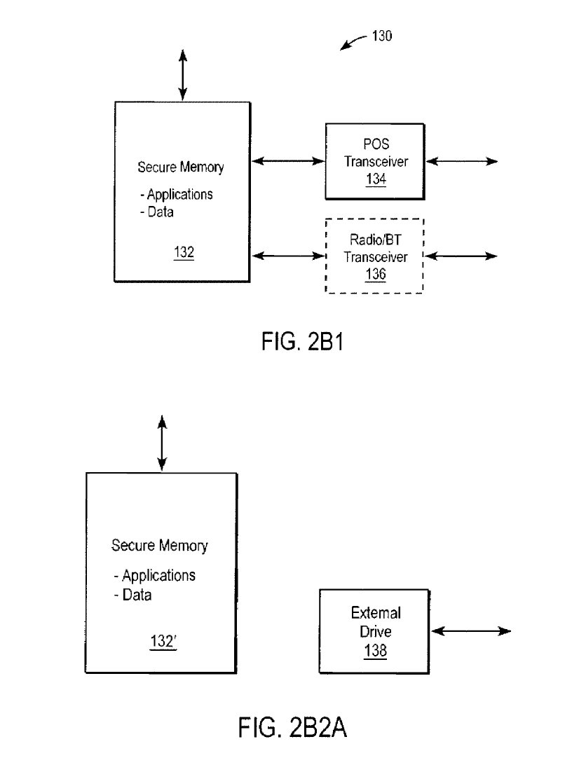

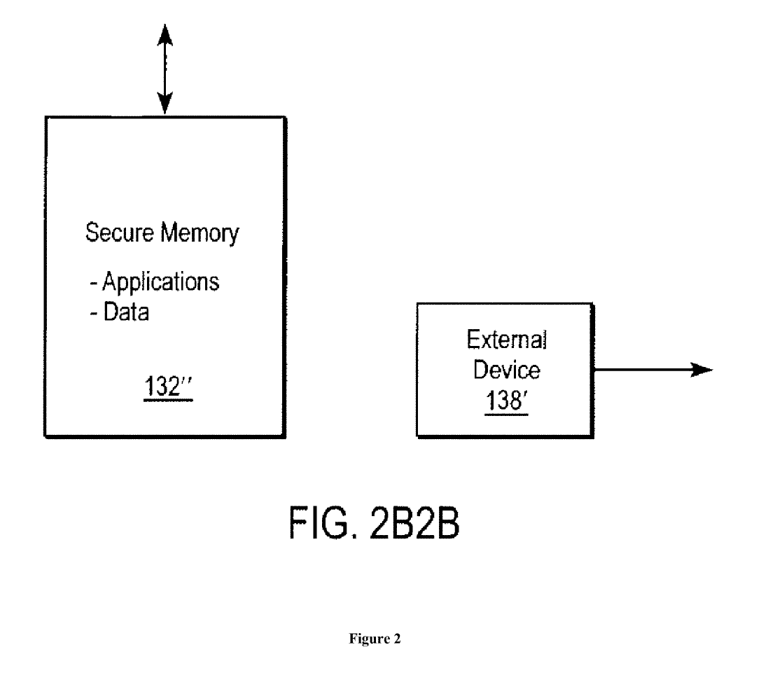

FIGS. 2B1, 2B2A, 2B2B illustrate more details regarding certain components illustrated in FIG. 1.

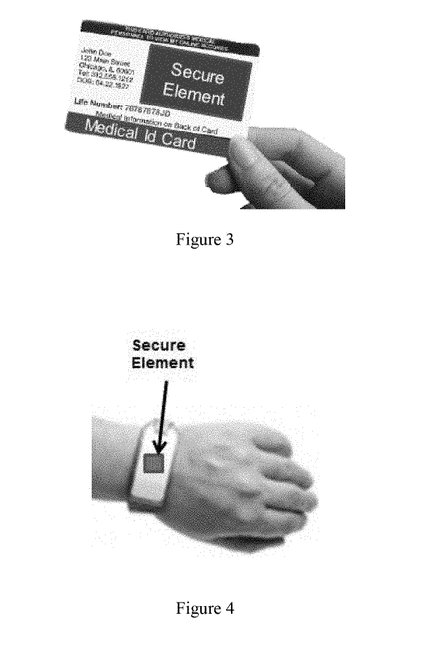

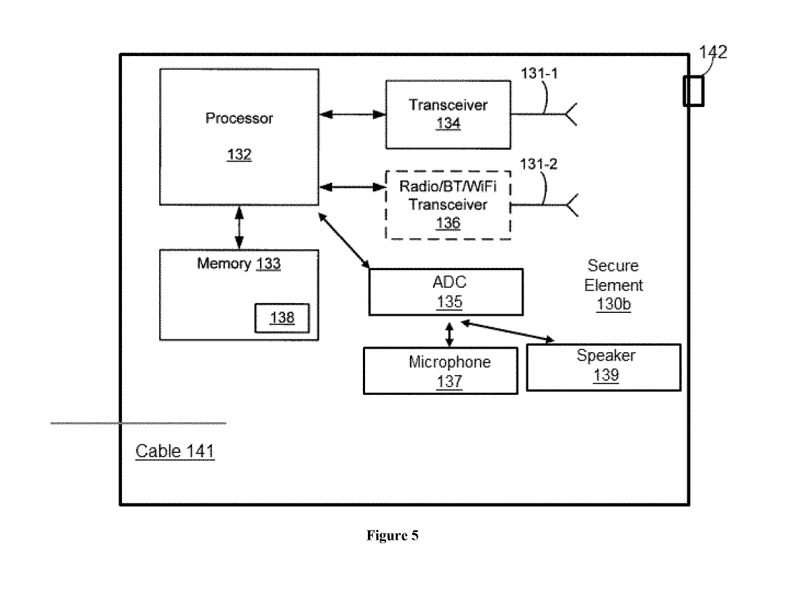

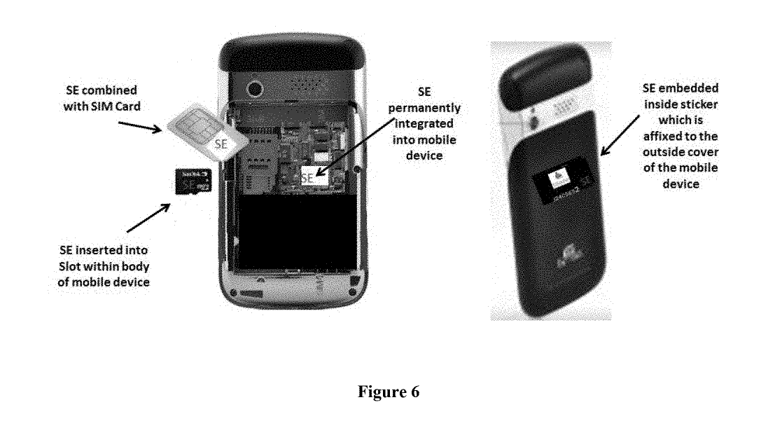

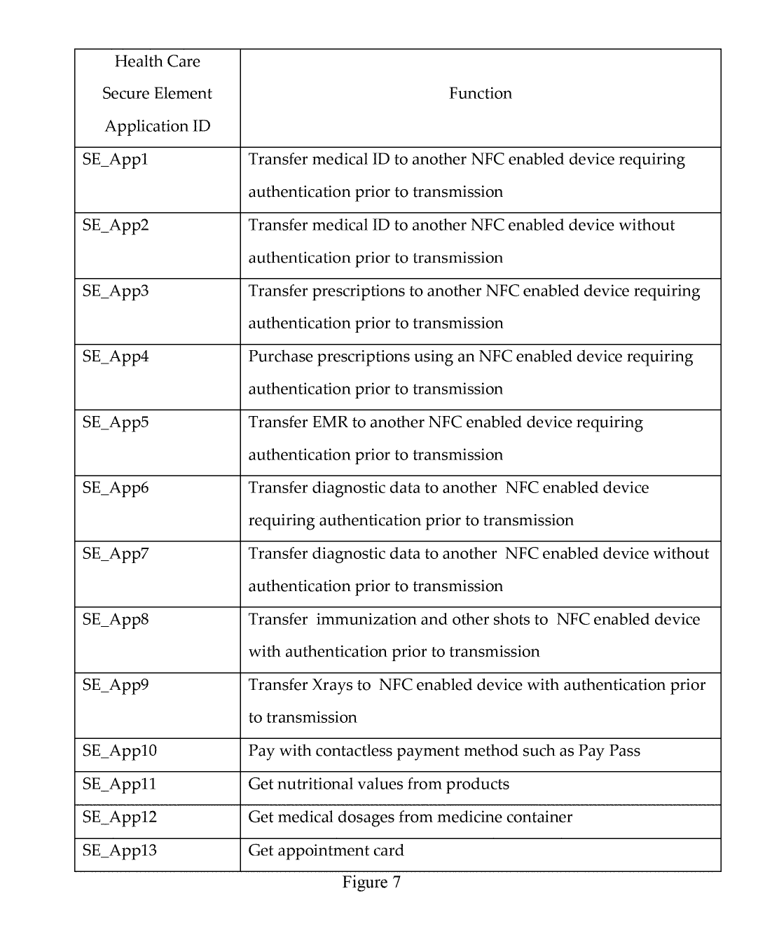

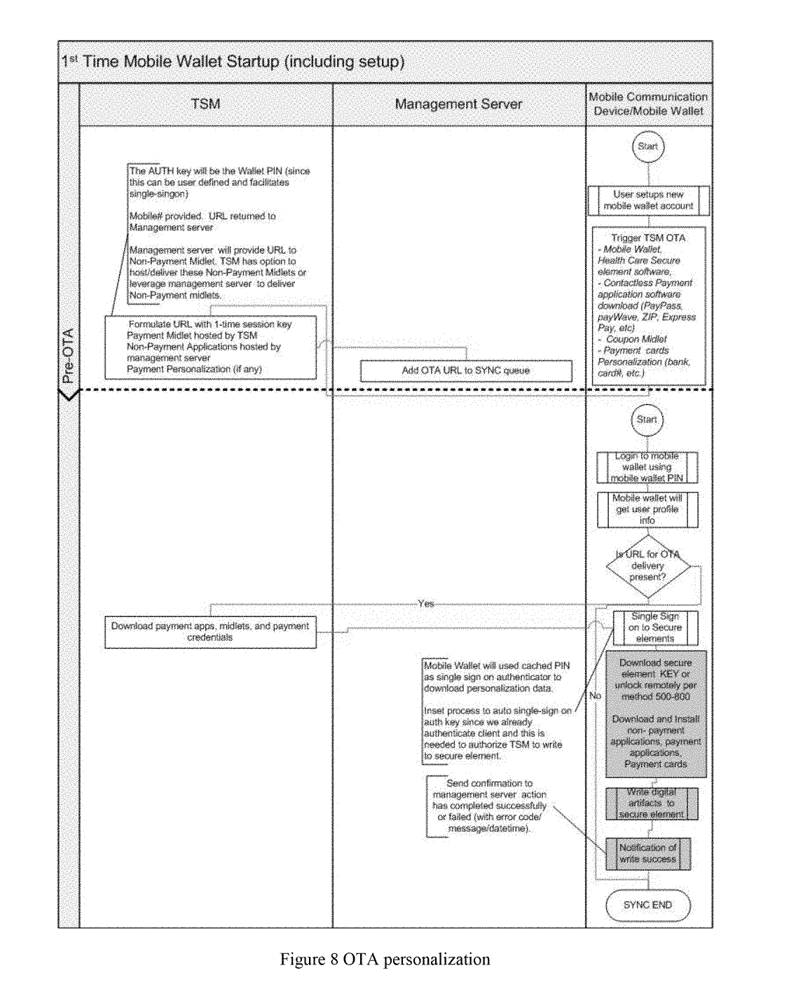

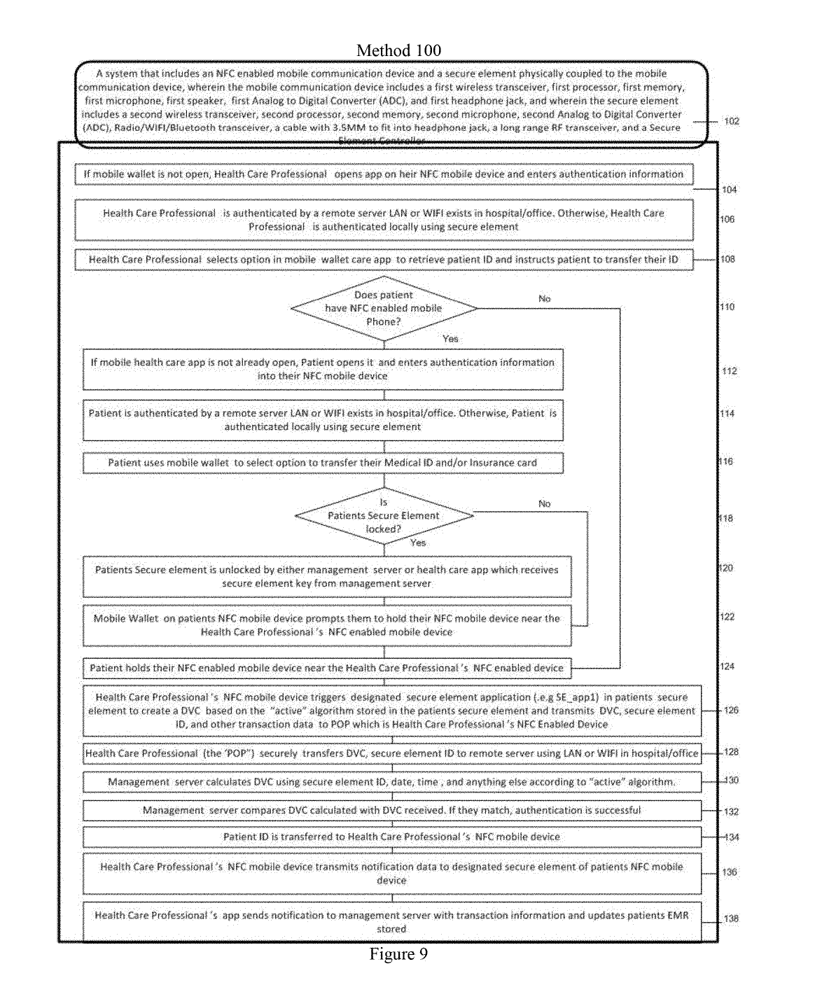

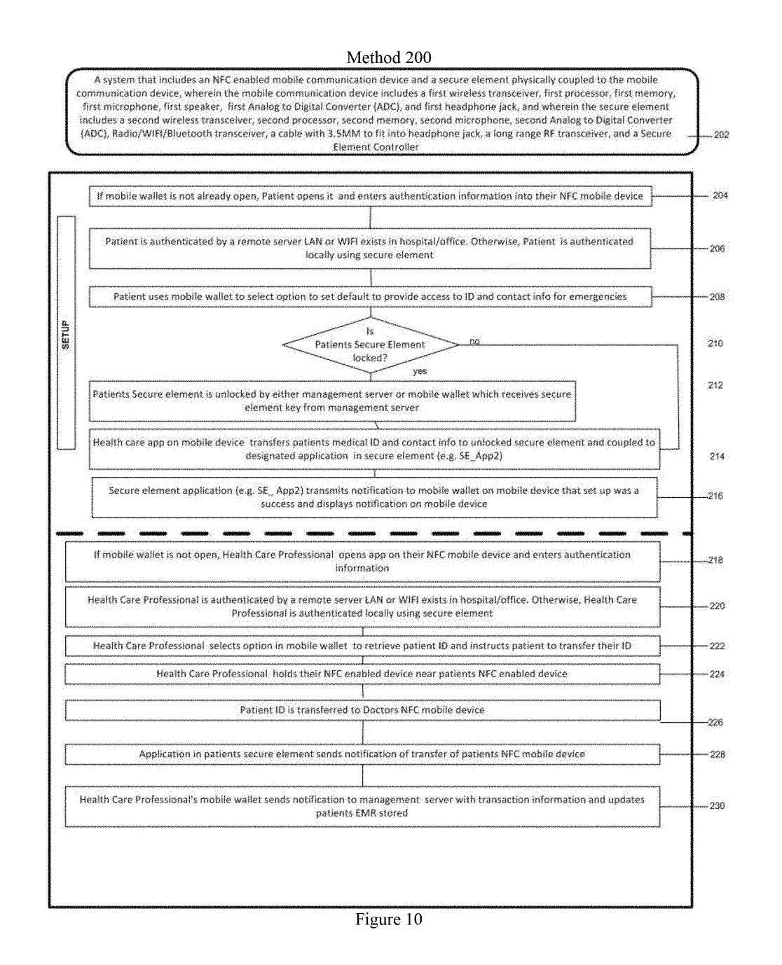

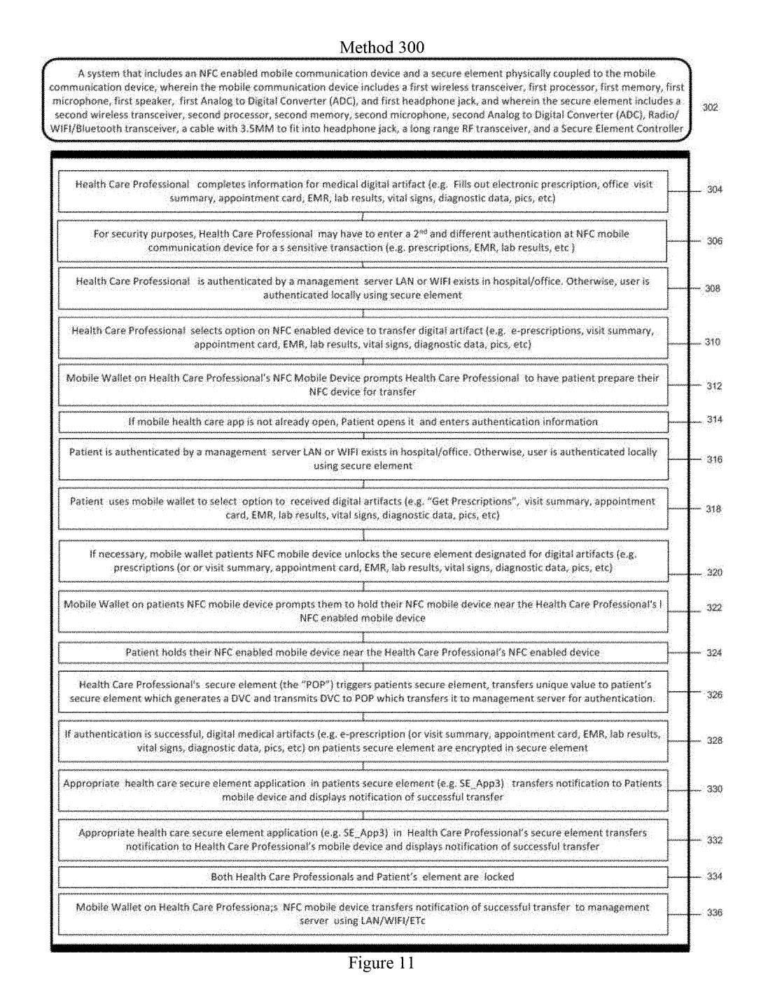

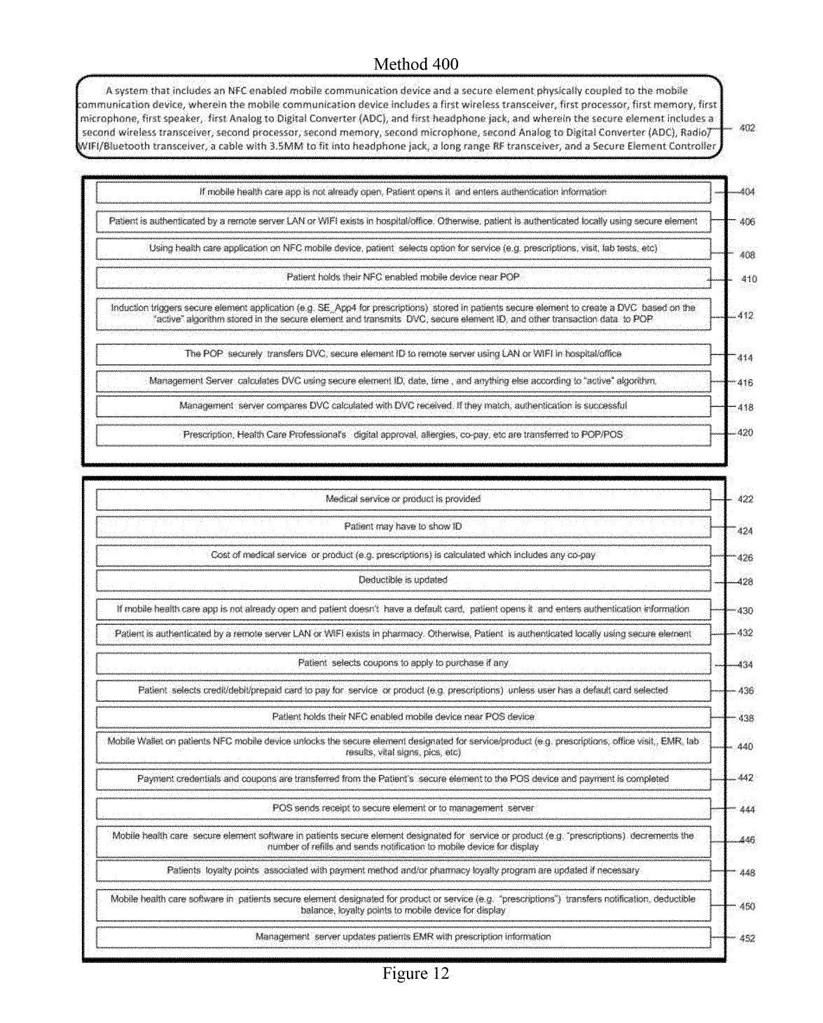

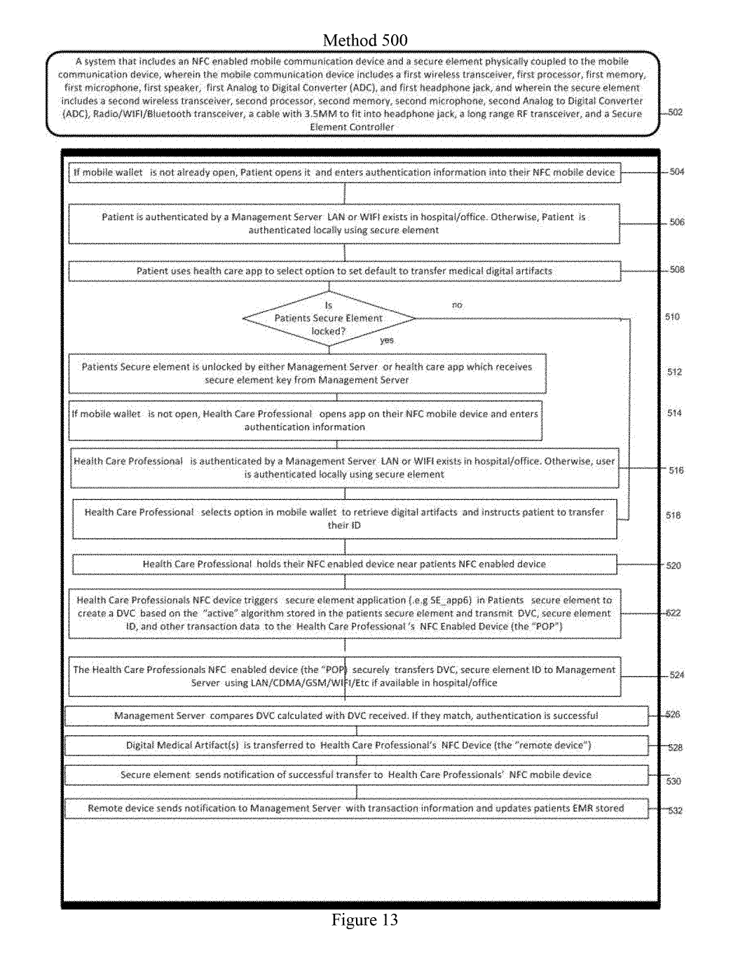

FIG. 3 illustrates a secure element embedded in an ID card

FIG. 4 illustrates a secure element embedded in a wristband

FIG. 5 illustrates more details regarding certain components of a secure illustrated in FIG. 1 of a secure element that is capable of bi-directional communication with a mobile device and transfer of data to a remote server

FIG. 6 illustrates multiple secure elements coupled to a mobile communication device

FIG. 7 illustrates secure element applications

FIG. 8 illustrates mobile wallet setup

FIG. 9 illustrates transferring a medical ID with authentication.

FIG. 10 illustrates transferring a medical ID without authentication.

FIG. 11 illustrates transferring a prescription

FIG. 12 illustrates paying for a prescription.

FIG. 13 illustrates transferring digital artifacts to a physician's NFC enabled mobile device

### DETAILED DESCRIPTION OF PREFERRED EMBODIMENTS

Reference will now be made in detail to various embodiments, examples of which are illustrated in the accompanying drawings. In the following detailed description, numerous specific details are set forth in order to provide a thorough understanding of the present inventions. However, it will be apparent to one of ordinary skill in the art that the present inventions may be practiced without these specific details. In other instances, well-known methods, procedures, components, and circuits have not been described in detail so as not to unnecessarily obscure aspects of the embodiments.

An NFC enabled device is one that includes a secure element and enables contactless transactions which have been described in more detail in previous patents including U.S. patent application Ser. No. 13/184,246, entitled “Using a mobile device to enable purchase of deals and increase customer loyalty” filed on Jul. 15, 2011, U.S. Provisional Patent Application Number 61/445,667, entitled “Non-Wireless Bidirectional Communication Between a Mobile Device and Associated Secure Element using an Audio Port” filed on Feb. 23, 2011 and U.S. Provisional Patent Application No. 61/429,246, entitled “Wireless Bidirectional Communications between a Mobile Device and Associated Secure Element using Inaudible Sound Waves,” filed Jan. 3, 2011 both of which are a continuation in part of U.S. patent application Ser. No. 12/948,717, entitled “Wireless Bidirectional Communications between a Mobile Device and Associated Secure Element,” filed Nov. 17, 2010, U.S. patent application Ser. No. 12/592,581 entitled “Method and Apparatus For Completing A Transaction Using A Wireless Mobile Communication Channel and Another Communication Channel”, filed Nov. 25, 2009, U.S. patent application Ser. No. 11/948,903, entitled “Method And System For Conducting An Online Payment Transaction Using A Mobile Communication Device” filed on Nov. 30, 2007, U.S. patent application Ser. No. 11/939,821, entitled “Method and System for Securing Transactions Made Through a Mobile Communication Device” filed Nov. 14, 2007, and U.S. patent application Ser. No. 11/933,351 entitled “Method and System for Purchasing Event Tickets Using a Mobile Communication Device”, filed Oct. 31, 2007, and U.S. patent application Ser. No. 11/933,321 entitled “Method and System for Adapting a Wireless Mobile Communication Device for Wireless Transactions,” filed Oct. 31, 2007 all of which are a continuation-in-part of U.S. patent application Ser. No. 11/467,441, entitled “Method and Apparatus for Completing a Transaction Using a Wireless Mobile Communication Channel and Another Communication Channel,” filed Aug. 25, 2006, which claims priority to U.S. Provisional Patent Application Nos. 60/766,171 and 60/766,172, both of which were filed Dec. 31, 2005. All of the above-referenced patent applications are incorporated by reference herein in their entirety.

As previously described in U.S. patent application Ser. No. 12/592,581, entitled “Method and Apparatus For Completing A Transaction Using A Wireless Mobile Communication Channel and Another Communication Channel”, filed Nov. 25, 2009 which is a continuation in part of U.S. patent application Ser. No. 11/467,441, entitled “Method and Apparatus For Completing A Transaction Using A Wireless Mobile Communication Channel and Another Communication Channel”, filed Aug. 25, 2006, FIG. 1 is a block diagram illustrating a communication system 100 in accordance with some embodiments. The communication system 100 includes a hand-held, wireless mobile communication device 110 with an antenna 120 for wireless communication. While the antenna 120 is shown as extending from the mobile communication device 110 for visual clarity, the antenna 120 may be implemented internally within the mobile communication device 110. Also, the mobile communication device 110 may include more than one antenna 120. The mobile communication device 110 includes a user interface for entering data. For example, a display 124 (FIG. 2) is a touch-screen display; alternatively or in addition, the mobile communication device 110 includes a keypad 125 (FIG. 2) for entering data.

A preferred embodiment, both the radio element 120 and the secure element 130 are both disposed internally within a body of the mobile device 110 as illustrated in FIG. 1, although in certain embodiments the secure element 130 can be external to the mobile device 110 as described hereinafter. In a variant implementation, the mobile device 110 contain a slot, which allows for the insertion of a secure element 130 into a slot and thus the physical insertion, mechanical and electrical connection as needed. In this configuration, the secure element can be purchased independently of the mobile device 110. The secure element 130 can also be disposed into a slot that only provides for physical insertion and mechanical connection to the body of the mobile device 110, and can then preferably include a transceiver that allows for the communication with the radio element 130 using a wireless local communication channel.

The radio element 120 also is illustrated as optionally including another transceiver 129, such as a Bluetooth or WIFI transceiver, which can transmit and receive signals with an external device and then communicate signals to and from the radio processor 123. This additional communication channel allows for communications between other external devices, such as an external Bluetooth enabled smartcard, and provides an additional communication channel that is useful for certain transactions, as described further herein.

FIG. 2B1 illustrates a preferred embodiment of the secure element 130 associated with the mobile device 110, the secure element 130 being commonly known as a smart card. As illustrated, the secure element 130 has a secure processor 132, a secure memory 133 and a POS/NFC transceiver 134 adapted to send transaction request signals and receive transaction response signals over a first communication channel. The secure processor 132 communicates via the secure element driver 128 with the radio processor 123, using, as mentioned above, signals described in the Java Card 2.1 Platform API Specification. The transaction request signals and the transaction response signals associated with the transaction preferably include identification code associated with the user, as well as information relative to the transaction, such as item, quantity, vendor, as is known. The POS transceiver 134 is preferably an NFC device of some type, which uses an NFC modem, although it can also be a Bluetooth, WIFI or other transceiver. In the case of the implementation of the POS transceiver being an NFC modem, such an NFC modem will typically have a set of registers that can be read/written by the secure processor 132. These registers are in turn available for reading and writing over the RFID communications channel and serve as a sort of shared memory between the secure processor 123 within the secure element 130 and the RFID reader that is associated with the POS terminal 150. This communication is specified, for example, in the ISO 14443A/B standard and ISO 15693 just to name a few.

Illustrated in an alternative embodiment is the inclusion of a radio/Bluetooth/WIFI transceiver 136, which can communicate with other devices, such as a transceiver associated with the radio processor 120 or for other external devices having those communication capabilities, thus allowing for more flexibility.

FIG. 2B2A shows a modified secure element 130, in which the radio element 120 does not communicate with the secure element through a secure element driver 128 of some type. In this case, for example, the secure element 130 may be external to the mobile device 110 and as such is not connected to the radio element through the secure element driver 128. In such an implementation, however, if the transceiver 136 as described above is included, and a similar transceiver 129 associated with the radio element 130 as described previously with respect to FIG. 1 is included, then this communication channel can be used to wirelessly obtain direct communications between the radio element 120 and the secure element 130.

This implementation allows for certain bidirectional communications with other devices, as well as with the radio element 120, and as such more functionality and flexibility is achieved. This implementation is particularly useful since it establishes a direct local communication path with the radio element 120, since there is not communications with the radio element 120 via the path of driver 128.

If either of the transceivers 129 or 136 are not associated with the respective radio element 120 or secure element 130, and there is no direct connection between the radio element 120 an the secure element 130, then a direct communication link between the radio element 120 an the secure element 130 will not exist. As such, while ticketing and many transactions can still exist, data from a real-time transaction performed using the secure element 130 cannot be made directly available to the radio processor and the applications stored thereon, which can prevent, for example, certain redundancy checks to occur, such as a ticketing application in which, after the ticket order has been placed, the ticketing application in the memory 126 associated with the radio element 130 can be programmed to provide an alert if the ticket receipt, via the management server 180, has not been received within a certain period of time. Such an alert would not be possible to program directly (although it could be programmed indirectly via the button panel on the phone, though such an implementation requires extra user intervention, which, if possible, one attempts to avoid in transactions such as this.

FIG. 2B2B shows a modified secure element 130″, in which the secure element does not include a processor that is capable of bidirectional communications, but instead is a passive device 138′, such as an RFID sticker or some other tag, that allows for a user identification, such that a transaction that is initiated with the passive device 138′ will cause the management server 180 to perform the transaction details. In this implementation, the code received from the POS terminal 150 or the POE terminal 190 is transmitted from the POS terminal 150 or the POE terminal 190 to the management server 180, which then takes over the transaction. This passive device 138′, with the identification code stored thereon, can thus be associated with a mobile device 110 not otherwise equipped for such communications, and the management server 190 can provide transactional information to the mobile device 110 using available channels on the mobile device (such as audio, sms or other known data transmission methods). While bidirectional communications do not occur with other devices, transactions are possible, because the management server 190 is involved.

FIG. 2B2C illustrates a secure element that is capable of either two-way communication or one-way communication with a POS/POE device that is not affixed to a mobile device nor electrically coupled to the mobile device. Instead the secure element is affixed to a hospital bracelet. FIG. 3 illustrates a secure element that is affixed to a affixed to a plastic medical ID card and FIG. 4 illustrates a secure element affixed to a affixed to a hospital bracelet, for example. Similarly, a secure element that is not affixed to a mobile device nor is electrically coupled to a mobile device can be incorporated with a key chain, watch, ring, bracelet worn as jewelry.

As illustrated in FIG. 5, and enhanced secure element 130 that is capable of bi-directional communication with a mobile device has a secure NFC processor 132, a secure memory 133 and a POS/NFC transceiver 134 adapted to send transaction request signals and receive transaction response signals over a first communication channel. A/D converter 135, a microphone 137, a speaker 139, and a, cable 141, power harvester, 144, Long range RF transceiver, 146, and a SE controller, 148. The Secure Element Controller 148 can either be logical or physical. In a logical implementation, it simply utilizes the primary secure element transceiver 134, memory 133 with data and applications, and a processor 132 to communicate with multiple logical or physical secure elements. Stated another way, the Secure Element Controller is the parent controller and all of the other logical or physical secure elements. The Secure Element Controller can issue read/write commands to all of the other logical or physical secure elements.

As described above, the secure element 130 has a secure processor 132, a secure memory 133 and a POS or NFC transceiver 134 adapted to send transaction request signals and receive transaction response signals over a first communication channel. In some embodiments a secure element 130 is internal to the mobile communication device 110. In some embodiments, the secure element 130 is externally attached to the mobile communication device 110. For example, the secure element 130 is adhesively affixed or mechanically secured to the housing of the mobile communication device 110. Alternatively, the secure element 130 is housed within a slot in the mobile communication device 110. The secure element 130 includes an antenna 131 for wireless communication. While the antenna 131 is shown as extending from the secure element 130 for visual clarity, the antenna 131 may be implemented internally within the secure element 130. Also, the secure element 130 may include more than one antenna 131. Communication occurs wirelessly between the secure element 130 and the mobile communication device 110 via respective antennas 120 and 131, over a direct wireless channel 163 between the mobile communication device 110 and the secure element 130. Thus, in some embodiments, the channel 163 does not pass through a network.

The point of sale terminal 150 illustrated in FIG. 1 is conventional, in that it has the capability of electronically reading information from a device equipped to transmit information in a format that it reads. Thus, the reader (not shown) within the point of sale terminal 150 can be of one or many types. If the point of sale terminal reader (not shown) includes the provision for NFC communications, then simply bringing the secure element 130 with the NFC transceiver will cause initiation of a transaction and the transmission of the identification code associated with the secure element 130 and thus the user.

For the embodiments described above, various software that is downloaded into the memory 126 of the radio element 120 and the secure memory 132 of the secure element 130, along with software resident on the management server 180, will cooperate at a layer that is above the physical layer of the communications, in order for the desired transaction to occur. This software is implemented using based upon known knowledge of mobile device 110 internals and application platforms, NFC, smartcard internals and application platforms, payment protocols (e.g. PayPass), and the working/workflow associated with POS and POE terminals, and the transaction and management servers. In addition, the present invention provides for piggybacking a tunneling protocol on top of the payment protocol, so that the secure elements 130 can communicate with the transaction server 170 and/or the management server 180, without modification to the POS terminal 150 or the POE terminal 190. As such, this includes software within the secure element 130 that embeds the required information in fields which will not adversely affect the performance of the POS terminal 150 and/or the POE terminal 190, and also software in transaction server 170 that will extract the piggybacked payload, associate the payload with the management server 180 if needed, and then authenticate, authorize, and execute transfers of transaction information to the management server 180.

In another embodiment, the piggybacked payload is sent, instead of to the transaction server 170, to the management server 180, which can then associate the transaction and notify the transaction server 170, the POS terminal 150 and/or the POE terminal as needed.

There are several types of NFC transactions. First, when the secure element is held in proximity to a remote device (i.e. Point-Of-Sale (POS) device 150, Point-Of-Entry (POE) 190, Point-Of-Presence (POP) device 200, smart poster, another secure element, etc) the POS/POE/POP activates or powers the secure element 130 through induction and triggers an application residing in the secure element (which is different than an application residing on the mobile communication device. So, the secure element need not have its own power source nor does the secure element require use of the battery inside of the mobile device. Stated another way, if the battery in the mobile device is dead, the user may not be able to use the mobile device to place a phone call, receive an SMS, or surf the web, but they will be able to purchase item and receive coupons and other digital artifacts using the secure element since the secure element is powered by the remote NFC enabled POS/POE/POP device. To reiterate, after triggering the secure element, an application residing in the secure element (which is different than an application residing on the mobile communication device) is launched and the application can transfer digital artifacts to the POS/POE/POP and the POS/POE/POP terminal can transmit digital artifacts to the secure element again even if the battery inside of the mobile communication device lacks power. There are instances when the NFC enabled mobile device is active and it can power any NFC device in passive mode including a POS/POE/POP, smart poster, or another NFC enabled mobile device which will described in more detail further herein.

While a POS may receive payment credentials from the secure element which is processed by the transaction server 170, the POS may transfer a coupon, receipt, or some other digital artifact to the secure element simultaneously during the induction process from the POS terminal 150, the transaction server 170 or the management server 180. Stated another way, the user need only hold, “wave” or “tap” their NFC enabled device once near the POS and both the transmission of the ticket to the POP and the receipt of the digital artifact will occur simultaneously so the user will not have to “tap” or “wave” their phone twice. This is accomplished through induction as well. The POS device/terminal will activate the secure element and an application residing in the secure element (which is different than an application residing on the mobile communication device) will facilitate the transfer.

As mentioned previously a Point-Of-Entry (POE) terminal is also equipped with NFC. When a user holds, “waves”, or “taps” their NFC enabled device near a POE terminal, the POE terminal activates the secure element in the NFC enabled mobile device and triggers an application in the secure element to transfer a ticket, for example, to the POE device which enables the user to gain access to the venue, building, automobile, etc.

While an NFC enabled Point of Sale (POS) facilitates a contactless purchase or financial transaction and a Point-of-Entry (POE) terminal facilities contactless access, an NFC enabled Point of Presence (POP) facilitates contactless transaction. A POP device is similar to an NFC POS enabled device in that it does contain a secure element and is capable of NFC communications (including two way communications). A POP is also capable of induction to be able to power an NFC enabled mobile communication device held in proximity to it and triggers an application running in the secure element to transfer information from the NFC enabled mobile communication device to the POP. Some examples of POPs include a kiosk, automobile, hotel, house, smart poster, smart catalogue, computer, medical device, another NFC mobile device, a music player, products at a retail store, etc. One example is a user holds their NFC enabled device near a kiosk which powers the secure element and launches an application to transmit a free song from the kiosk to the secure element. The POP can send/receive transaction data to the management server, 180, have transactions processed by the transaction server 170, or transfer/receive digital artifacts to/from the secure element 130 at the remote server 210.

A POP may be a Smart Poster—When an NFC enabled mobile communication device is held in proximity to a smart poster, the smart poster may transmit information, data, maps, or any other type of digital artifact to the secure element.

A POP may be a computer, laptop, tablet, etc—. A user first uses the mobile wallet to select which items to be transferred to the POP and instructs the user to hold the NFC device near the POP. When an NFC enabled mobile communication device is held in proximity to the computer, the computer can transmit information, data, maps, or any other type of digital artifact to the secure element

A POP may be another NFC Enabled Mobile communication device. A user first uses the mobile wallet to select which items to he transferred to the POP and instructs the user to hold the NFC device near the POP. When an NFC enabled mobile communication device is held in proximity to another NFC enabled device may transmit contact information, email, web site addresses, social media information, tickets, coupons, receipts, vouchers, content, or any other type of digital artifact to the secure element

As described in U.S. patent application Ser. No. 13/213, 840 entitled “Transferring Data Between NFC Enabled Mobile Devices With Multiple Secure Elements” filed on Aug. 19, 2011 which is incorporated herein by reference, after a user holds an NFC enabled mobile device in proximity to an NFC POS/POE/POP, the NFC POS/POE/POP can transfer secondary digital artifacts to the secure element such as Xrays, tickets, business cards, music, product information, etc. As described in more detail in the patent applications mentioned above, a user may have to authenticate and the secure element may have to be unlocked prior to the storage of digital artifacts in the secure element. For security purposes, the secure element keys are located at the management server, 180 and may be managed by an entity sometimes referred to as a “Trusted Server Manager”. In cases where network availability is not reliable and performance is critical, the secure element key may be downloaded to the mobile device in an encrypted file. This communication is specified, for example, in the ISO 14443A/B standard and ISO 15693 just to name a few

As mentioned previously, an NFC enabled mobile communication device may have multiple secure elements. There may be a secure element may be dedicated to specific financial transactions vs another secure element which may be dedicated to medical records and have a separate secure element key. There may even be a secure element that is “public domain” which any POS or device can write to without any restricted accesses. As shown in FIG. 7, there can be a different “health care secure element software application; for different functions. There may be 1 logical secure element or multiple secure elements as described in U.S. patent application Ser. No. 13/213,840, entitled “Transferring Data Between NFC Enabled Mobile Devices With Multiple Secure Elements” filed on Aug. 19, 2011. There may be a secure element and application for prescriptions, office visits, appointments, EMR, pictures, etc. A user may only need to tap once to transfer artifacts to multiple secure elements. Alternatively, a user may tap once to transfer an ID, for example, from the secure element to the POP and then tap another time to transfer artifacts from the POP such as a prescription from the POP to the secure element. The transfer of digital artifacts between secure element and a POP is a result each application stored in the secure element having its own Id. So for example, a secure element application that facilitates transfer of a patient's medical ID to/from a secure element may have an ID of “SE_App1. In contrast, a secure element application that facilitates transfer of prescriptions to/from a secure element may have an ID of “SE_App3”. Whenever multiple applications exist in the secure element, the applications must be prioritized so that the POP/POS/POE will know which application to trigger and in which order. So, for example if the user selects to get a prescription using their mobile wallet, the mobile wallet will transfer a signal to the secure element to make the “X ray secure element application or SE_App3” the active one and assign it priority of 1 so that the POP/POS/POE will select it first. The mobile wallet communicates to the secure element in a number of different ways regardless of whether secure element is internal, inserted into a slot within the body of the mobile device, or affixed externally to the body of a mobile device as described in U.S. Provisional Patent Application No. 61/445,667, entitled “Non-Wireless Bidirectional Communication Between a Mobile Device and Associated Secure Element using an Audio Port” filed on Feb. 23, 2011 and U.S. Provisional Patent Application No. 61/429,246, entitled “Wireless Bidirectional Communications between a Mobile Device and Associated Secure Element using Inaudible Sound Waves,” filed Jan. 3, 2011 both of which are a continuation in part of U.S. patent application Ser. No. 12/948,717, entitled “Wireless Bidirectional Communications between a Mobile Device and Associated Secure Element,” filed Nov. 17, 2010 which are incorporated by reference herein

Prior to first use, a mobile wallet and health care secure element application(s) should either pre-installed on the mobile device and secure element respectively. Alternatively, they can be downloaded Over the Air (OTA) using conventional wireless as illustrated in FIG. 8, IR, or even USB cable loading through a personal computer. After the mobile wallet and health care secure element software are installed, the patient or user will have to activate the app either by typing in a one-time activation code into the mobile app wherein they receive via U.S. Mail, text messaging, email, or their mobile device. Alternatively, the user can activate the app by calling an 800# and providing the activation code. Use will have to select a “PIN” for their mobile wallet which is authenticated by a remote server. PIN can be a numerical value, alpha numerical value, biometric (e.g. fingerprint scan, retina scan), Refer to U.S. patent Ser. No. 11/939,821, entitled “ Method and System for Securing Transactions Made Through a Mobile Communication Device”, filed Nov. 14, 2007.

The mobile wallet software residing on the mobile device will enable the patient to view their health plan benefits including participating physicians, hospitals, specialists, labs, medications covered, co-pay, and coverage for different treatments and prescriptions. The application can also provide background information on doctors, contact information, etc.

Similarly, health care professionals (nurse, doctor, pharmacist, etc) will download a mobile wallet and health care secure element application(s) if they are not pre-installed on the mobile device and secure element respectively. Alternatively, they can be downloaded Over the Air (OTA) using conventional wireless as illustrated in FIG. 8, IR, or even USB cable loading through a personal computer. After the mobile wallet and health care secure element applications are installed, the user will have to activate the app either by typing in a one-time activation code into the mobile app wherein they receive via U.S. Mail, text messaging, email, or their mobile device. Alternatively, the health care personnel can activate the app by calling an 800# and providing the activation code. Use will have to select a “PIN” for their mobile wallet which is authenticated by a remote server. PIN can be a numerical value, alpha numerical value, biometric (e.g. fingerprint scan, retina scan), Refer to U.S. Pat. No. 11/939,821, entitled “ Method and System for Securing Transactions Made Through a Mobile Communication Device”, filed Nov. 14, 2007.

In one embodiment as described in method 100, with a “wave” of their NFC enabled mobile device, a health care professional can “scan” the patients secure element and identify who patient is. Patients or users may have to provide permission prior to having their ID scanned. The health care professional opens their mobile wallet and enters their authentication information (104). They are authenticated by the management server (106). Afterwards, they select an option in their wallet to obtain a patients ID (108). If the patient has an NFC enabled phone (110), they will open their wallet and enter authentication (112) information (e.g. PIN or biometrics such as finger print scan) and is authenticated by the management (114) if WIFI or wireless service is available at the medical facility. Otherwise, the patient can authenticate using PIN stored in the secure element. If a patient has a medical ID card with a secure element, medical bracelet with a secure element, or some non-mobile device, they will skip this step. The patient uses the mobile wallet to select the option to transfer their medical ID (116). If the secure element is unlocked (118), it's unlocked by the management server or the mobile wallet (120). Upon doing so the mobile wallet application will transfer a signal to the secure element to make the “medical ID secure element application that requires authentication or SE_App1” the active application in the secure element or assign it priority of so that the POP/POE/POS will select it first. If there are multiple secure elements, the secure element controller which maintains a directory of the location of secure elements and applications will direct the POP/POS/POE to the location of the active secure element application. The mobile wallet prompts (122) the patient to hold their NFC enabled device near the health care professionals NFC enabled device which the patient does (124). The health care professionals' NFC enabled mobile device functions as a POP and powers the patients NFC enabled device through induction. For security and verification purposes, the POP transmits a random value to the patients secure element and triggers the appropriate secure element application (e.g. SE_App1) in the patients NFC enabled NFC device to create a DVC (126) using the random value from the POP. The health care professionals NFC enabled mobile device (the “POP”) transfers the DVC and other transaction data to the management server (128). The management server calculates a DVC (130) using the transaction data and verification algorithm stored in the secure element and compares the calculated value with the received DVC (132). If they match, authentication is successful and the management server notifies the health care physicians NFC enabled device (the “POP”). The secure element application (e.g. SE_App1) transfers the patient's medical ID to the health care physicians NFC enabled mobile device (134). One of the benefits of this approach is that if a patient changes health care providers and gets assigned a new medical ID, they can update the information in the secure element without getting a new NFC enabled phone even if they have an “NFC smart sticker” which is secure element capable of two transmission, their medical ID, basic patient information, emergency contact information, etc using their mobile device and not have to get a new “smart sticker”.

In another embodiment as illustrated in method 200, with a “wave” of their NFC enabled mobile device, a health care personnel can identify who patient is without requiring the patients authorization if the patient has configured their NFC device to do so (204-216). During the configuration, the mobile wallet application will transfer a signal to the secure element to make the “medical ID secure element application that does not require authentication or SE_App2” listed in FIG. 7 the active application in the secure element or assign it priority of so that the POP/POE/POS will select it first. If there are multiple secure elements, the secure element controller which maintains a directory of the location of secure elements and applications will direct the POP/POS/POE to the location of the active secure element application. The Health Care Professional (i.e. nurse, doctor, pharmacist, etc) opens their mobile wallet (218) and enters authentication information (i.e. PIN, fingerprint scan, retina scan) at their mobile device (220). After authentication, the health care professional selects an option to get patient ID (222). Then they hold their NFC enabled device near the patients (224). Health Care Professional's NFC mobile device triggers (226) designated secure element application (e.g SE_app2) in patients secure element to create a DVC based on the “active” algorithm stored in the patients secure element and transmits DVC, secure element ID, and other transaction data to POP which is Health Care Professional's NFC Enabled Device. Application in patients secure element sends notification of transfer of patients NFC mobile device (228) Health Care Professional's mobile wallet sends notification to management server with transaction information and updates patients EMR stored (230).

The Health Care Professionals (nurse, doctor, pharmacist, etc) can transfer medical digital artifacts to a patients NFC enabled device. Such medical digital artifacts include, but are not limited to a medical ID, basic patient information, patient contact information, emergency contact information, primary care physician information, health insurance information including co-pay and deductibles, prescriptions, office visit summary, appointment cards, Electronic Medical Records (EMR), lab results, blood type, organ/donor status, vital signs, diagnostic data, immunization records, payments and transaction history, pictures, etc.

For example, a Health Care Professional can transfer EMR records to/from their NFC enabled mobile device with a “wave” of a patients NFC enabled mobile device. In method 300, its assumed that the Health Care Professional has already authenticate initially and scanned the patients' medical ID before retrieving the patients EMR using methods described above. The Health Care Professional first writes a prescription, schedule an appointment, transfer medical records, etc, using their mobile wallet application (304). For sensitive transactions such as prescriptions, they may be required to provide a 2^{nd} form or multifactor authentication (306) and authenticated by either the management server or locally (308). The health care professional will select the transaction they want to perform such as transfer EMR (310) ask the patient to prepare their NFC enabled mobile device to receive the digital artifact (312). If the patient's mobile wallet is not open, they will open it and enter authentication information (i.e. PIN, fingerprint scan, retina scan) (314) where authentication is performed by a remote server (316). If WIFI or wireless carrier connectivity is not available at the hospital or medical facility, the patient can authenticated locally with an ID stored in the secure element such as a finger print scan or PIN. The patient will then select an option to receive the digital artifacts (318). If necessary, the patients secure element is unlocked (320) using techniques described in U.S. patent Ser. No. 13/213,840, entitled “Transferring Data Between NFC Enabled Mobile Devices With Multiple Secure Elements” filed on Aug. 19, 2011. During this process, the mobile wallet application will transfer a signal to the secure element to make the “Transfer EMR secure element application which require authentication or SE_App5” listed in FIG. 7 the active one or assign it priority of 1 or make it the active secure element application so that the POP/POS/POE will select it first. If there are multiple secure elements, the secure element controller which maintains a directory of the location of secure elements and applications will direct the POP/POS/POE to the location of the active secure element application. The mobile wallet on the patients NFC enabled mobile device will prompt (322) them to hold their device near the Health Care Professional's NFC enabled mobile device. When the patient does this (324), the Health Care Professionals NFC enabled mobile device activates or powers the patients secure element. For sensitive digital artifacts such as lab results, x-rays, electronic medical records, prior to transmission of the digital artifacts, the patient and mobile device are verified using the dynamic verification code (326) method described in U.S. patent Ser. No. 13/213,840, entitled “Transferring Data Between NFC Enabled Mobile Devices With Multiple Secure Elements” filed on Aug. 19, 2011. The digital medical artifacts are transferred from the Health Care Professional's secure element and encrypted at the patient's secure element (328). After transfer, the Patients secure element sends a notification to the patients mobile wallet (330) and the Health Care Professional's secure element sends a notification to the their mobile wallet (332). Both the patient and health care professional secure elements are locked (334). The Health Care Professionals' mobile wallet also sends a notification of the transaction to the management server and updates the patients EMR which is stored at the management server or in the “cloud” (336).

Another example that lends itself well to busy parents who need to track their children's vaccinations for measles, mumps, chickenpox, etc. Following the steps in Method 300, the Health Care Professional can record the shots on a NFC enabled mobile device (e.g. such as an iPad) and transfer the information about the shots including the dates, dosage, etc to the parents NFC enabled mobile device simply by bringing them in proximity to each other. Both the health care professional and the parent have to open their mobile wallets, authenticate, and select the option to transfer the data regarding the medical shots. During this process, the mobile wallet application will transfer a signal to the secure element to make the “Transfer immunizations secure element application which require authentication or SE_App8” listed in FIG. 7 the active application in the secure element or assign it priority of so that the POP/POE/POS will select it first. If there are multiple secure elements, the secure element controller which maintains a directory of the location of secure elements and applications will direct the POP/POS/POE to the location of the active secure element application. This way, parents can keep an electronic record of their children's vaccinations. If parents need to provide proof of these vaccinations to schools, they can transfer them via NFC induction to the school nurse by holding their NFC enabled device in proximity to the school staff's NFC enabled mobile device. In both cases, as illustrated in method 300, prior to transfer of records, the nurse/doctor will have to authenticate and receive confirmation from the parent and parent will have to authenticate prior to transferring records to school nurse. If the parent has a mobile wallet on their mobile device, the health care secure element app can provide a list of vaccinations by age group and a reminder notification for each of their children. Similarly frequent international travelers can have a record of their shots transferred via induction from the health care personnel's NFC enabled mobile device to their NFC enabled mobile device.

In a similar process, a patient can receive a digital appointment card from a Health Care Professional such as a nurse or assistant. Following the steps in method 300, the Health Care Professional fills out the digital appointment card. The patient opens their mobile wallet, authenticating, and selecting an option such as “Get Appointment Card”. During this process, the mobile wallet application will transfer a signal to the secure element to make the “Get appointment card secure element application which require authentication or SE_App13” listed in FIG. 7 the active one or assign it priority of 1 or make it the active secure element application so that the POP/POS/POE will select it first. If there are multiple secure elements, the secure element controller which maintains a directory of the location of secure elements and applications will direct the POP/POS/POE to the location of the active secure element application After doing so, the mobile wallet prompts the user to hold their NFC enabled device near the Health Care Professionals NFC enabled device (which could be an NFC phone, personal computer, tablet, etc). Upon doing this, the health care' professionals NFC Device which serves as the POP will trigger the secure element app (e.g. SE_App13) in the patients NFC enabled device and transfer the patients UserID and in return transfer the digital appointment card to the patients secure element. If the secure element is locked, it will be unlocked prior to the transmission of digital artifacts according to the steps in Method 300.

In another example, a Health Care Professional (nurse, doctor, pharmacist, etc) can transfer prescriptions to a patients NFC enabled device utilizing the procedures outlined in Method 300. A health care professional utilizes an NFC enabled device (e.g. an iPad or some other electronic tablet) and using their mobile wallet or similar application on the NFC enabled device select the patients prescription from a menu or list of available drugs and dosages, for example. This may mitigate scenarios where the doctor writing is difficult to read. Upon completing the “electronic prescription”, the health care professionals (nurse, doctor, pharmacist, etc) can hold the patients NFC enabled device near theirs and automatically transfer the electronic prescription via NFC induction to the patients NFC enabled device. During this process, the mobile wallet application will transfer a signal to the secure element to make the “Transfer prescriptions secure element application which require authentication or SE_App3” listed in FIG. 7 the active application in the secure element or assign it priority of so that the POP/POE/POS will select it first. The patient may have to authorize the transfer of the prescription first by pressing “accept” prescription in in their mobile wallet app running on their NFC enabled device. The doctor may also have to enter authentication at the mobile device prior to transmitting the prescription. The doctor's electronic prescription is synchronized with the patients electronic medical records which are stored in a remote server or cloud.

As illustrated in method 400, Patients can pay for prescriptions at CVS, Walgreens, Rite Aid, etc by “waving” of their phone near the POS terminal at the drug store. Also, prescriptions are automatically approved using list of allergies stored in the secure element of the NFC enabled mobile device and digital signature of physician. The secure element also contain a list of approved “over the counter” and “generic” medications that can be substituted for the prescription. After the pharmacist prepares the prescription, the pharmacist applies a label to the bottle. The label contains a bar code and/or RFID tag encoded with the prescription, date, time, pharmacy ID, Pharmacists ID, Doctor ID. The secure element can contain a list of the medications covered by their plan as well as the amount of coverage and co-pay. A patient opens their mobile wallet (404) if it is not already opened and enters authentication information at the mobile device (406) wherein they are authenticated by the management server. The patient selects prescriptions they want to fill (408) and holds) their NFC enabled mobile device near the POS/POP terminal at the pharmacy (410) During this process, the mobile wallet application will transfer a signal to the secure element to make the “Purchase prescriptions secure element application which require authentication or SE_App4” listed in FIG. 7 the active application in the secure element or assign it priority of so that the POP/POE/POS will select it first. If there are multiple secure elements, the secure element controller which maintains a directory of the location of secure elements and applications will direct the POP/POS/POE to the location of the active secure element application. The POP powers the secure element from the NFC induction and triggers the secure element application (i.e. SE_App4) to calculate a DVS and transfer it to the POP/PS (412) which is described in more detail is U.S. Pat. No. 13/213,840, entitled “Transferring Data Between NFC Enabled Mobile Devices With Multiple Secure Elements” filed on Aug. 19, 2011. The POP transfers he DVC to the management server (414) which calculates the DVC using the transaction data received and a similar verification algorithm stored in the secure element (416). The management compares the calculated DVC with the DVC it received (418). If they match, authentication is successful. The medical service (e.g. lab tests prescription filled, dental procedure, etc) (422). Depending on the medical service (e.g prescription filled), the patient may have to show ID (424). The health care professional calculates the cost of the medical service including co-pay unless the co-pay is calculated automatically when the information is transferred from the secure element to the POP. Since patient's medical ID and other transaction data are transferred from the secure element to the POP/POS terminal receives the amount of coverage which is treated as a coupon and deducts this from the total amount of the prescription. (424). The application in the secure element updates the deductible amount. The patient opens their mobile wallet and (430) and is authenticated again if necessary (432). Patient selects coupons for discounts on medications which may already be stored in the mobile wallet or secure element. If they are stored in the mobile wallet, they are transferred to the secure element (434). The patient selects payment method (436) and hold NFC enabled device near POS. During this process, the mobile wallet application will transfer a signal to the secure element to make the “Pay with contactless payment secure element application which require authentication or SE_App10” listed in FIG. 7 the active application in the secure element or assign it priority of so that the POP/POE/POS will select it first. If there are multiple secure elements, the secure element controller which maintains a directory of the location of secure elements and applications will direct the POP/POS/POE to the location of the active secure element application. When the patient holds. (438 “waves,” or “taps” their NFC enabled phone at the POS, the secure element is unlocked (440) coupons are automatically transferred to the POS and applied to the purchase, and the payment is made (442) with a single “wave” or “tap” of their NFC enabled mobile device. The patient pays for their prescription with the same single tap or multiple taps. After the patient pays for the prescription, the pharmacist scans either the bar code or the RFID tag and upon doing so, the information on the tag is “read” and transmitted to the mobile device secure element and transmitted to a remote server. The user may have to confirm that the pharmacist can “write” or transmit this info to the secure element prior to it be transmitted. The secure element is unlocked. POS sends receipt to secure element if possible and management server (444). The mobile health care secure element software in patients secure element designated for the service or product (e.g. “prescriptions) decrements the number of refills and sends notification to mobile device for display (446). Patients loyalty points associated with payment method and/or pharmacy loyalty program are updated if necessary (448). The mobile health care secure element software in patients secure element designated for product or service (e.g. “prescriptions”) transfers notification, deductible balance, loyalty points to mobile device for display (450). The management server updates patients EMR with prescription information (452).

In another embodiment, the patient can order their prescription or refill from their mobile wallet residing on their mobile device. The health care secure element application will transfer the prescription from the secure element to the mobile device for display. The patient can select the prescription they wish to fill. The patient select from a list of nearby pharmacies based on location of user using GPS. During this process, the mobile wallet application will transfer a signal to the secure element to make the “Purchase prescriptions secure element application which require authentication or SE_App4” listed in FIG. 7 the active application in the secure element or assign it priority of so that the POP/POE/POS will select it first. If there are multiple secure elements, the secure element controller which maintains a directory of the location of secure elements and applications will direct the POP/POS/POE to the location of the active secure element application. When the user finds a pharmacy they wish to use, they can use their mobile wallet to place the order for prescription electronically at the desired pharmacy. They may have to enter authentication information at the mobile device such as a PIN or biometrics (e.g. fingerprint) to authorize the prescription. Along with the prescription, the user also wirelessly transmit allergies and medical plan information which includes coverage amounts to the pharmacy as well as the encrypted doctors electronic approval to fulfill the prescription if the doctor transmitted it to the patient. Finally, the patient can select a payment method in their mobile wallet to pay for the prescription. If the patient has NFC and contactless payment software embedded into the touch screen of their NFC enabled device, they can utilize their NFC enabled device like a POS device and pay for their prescriptions using NFC induction. Alternatively, when they place the order electronically, they can authorize the pharmacy to charge a credit card or payment that the pharmacy has on file. For additional security and to prevent fraudulent prescriptions, the patient can configure their mobile wallet to restrict prescriptions to be filled at pre-identified pharmacists, limited to a certain number of refills, pharmacists within a certain geographical distance of the patients location, time of day, day of week, etc.

A patient can also utilize their NFC enabled device to order and pay for their prescriptions online. U.S. patent application Ser. No. 11/948,903 entitled, “Method And System For Conducting An Online Payment Transaction Using A Mobile Communication Device” filed on Nov. 30, 2007 describes various methods taught for facilitating online purchase using an NFC enabled mobile device. In one scenario, the patient uses their personal computer/tablet/laptop and a website to select and order the prescriptions they want to order or refill. At checkout on website, the patient can hold their NFC enabled device near an NFC POS device such as a personal computer equipped with NFC, a monitor equipped with NFC, a keyboard equipped with NFC. Alternatively, if patient user has an electronic prescription stored in the secure element of their NFC enabled device, the patient can hold their NFC enabled device near the POS (e.g. computer, monitor, keyboard, etc) and transfer the electronic prescription via NFC induction to the POS (e.g. computer, monitor, keyboard, etc). During this process, the mobile wallet application will transfer a signal to the secure element to make the “Purchase prescriptions secure element application which require authentication or SE_App4” listed in FIG. 7 the active application in the secure element or assign it priority of 1 so that the POP/POS/POE will select it first. If there are multiple secure elements, the secure element controller which maintains a directory of the location of secure elements and applications will direct the POP/POS/POE to the location of the active secure element application. The patients list of allergies, doctors digital signature, co-pay amount and any coupons also stored in the secure element are transferred via NFC induction using the same “single tap” of the NFC enabled phone or a secondary tap. Once it is determined that the prescription will not cause any reaction with known allergies and receipt of doctor digital approval, the online pharmacy calculates the total amount. The patient user may be prompted to hold their NFC enabled device near the POS again to transfer payment credentials unless a default card had been selected in which case this last “tap” is not necessary. The patient may have to enter authentication information either at the POS which is authenticated by a remote server or enter authentication information on the NFC enabled mobile device where authentication is done remotely. For additional security, prescriptions may only be filled at pre-identified pharmacists, number of refills, pharmacists within a certain geographical footprint.

The patient may elect to have their prescription mailed to them. When the delivery person arrives to deliver the prescription, the patient may have to manually sign to accept it. Alternatively, the patient may have to “tap” their NFC enabled device near the delivery persons NFC enabled device and upon doing so “signs” electronically for the delivery. In this step, the patient's ID is transferred from the secure element to the delver person's NFC enabled device (the “POP”) using NFC induction. The patient may also authenticate at their mobile device using a PIN or biometrics or a combination. In this case authentication is performed by a remote server. Upon successful authentication, the notification is transmitted wireless to the delivery persons NFC enabled mobile device. For extremely dangerous drugs, the prescription may be housed in a container that has an exterior digital lock with an embedded NFC chip. The patient must hold their NFC enabled device near the lock which utilizes the s patients ID or the patients secure element key to unlock the container using NFC induction. So, in this instance the secure element embedded in the lock is pre-encoded with a secure element key that the patient also has stored in their NFC enabled device. In either one of the steps described above, after the user signs for the package, a receipt is transmitted to the remote server and the patients EMR is updated with the information. Also, a receipt is transmitted to the secure element. The number of refills may be decremented. The next delivery of the prescription maybe scheduled and transmitted to the secure element or mobile device so that it can be displayed on a calendar integrated with the health care application.

In a preferred embodiment, a patient can configure their mobile wallet to display reminders of when medical bills are due such that they pop-up in their mobile wallet. When bills are due, they can elect to have them automatically paid using a payment method stored in their mobile wallet. Alternatively, the patient manually selects a payment method stored in their mobile wallet to pay for their bill. In yet another scenario, a user can tap and pay for their bill using their NFC enabled mobile deice at a point-of-sale device or kiosk. NFC contactless transactions are described in more detail in U.S. patent application Ser. No. 13/184,246, entitled “Using a mobile device to enable purchase of deals and increase customer loyalty” filed on Jul. 15, 2011, U.S. patent application Ser. No. 12/592,581 entitled “Method and Apparatus For Completing A Transaction Using A Wireless Mobile Communication Channel and Another Communication Channel”, filed Nov. 25, 2009, U.S. patent application Ser. No. 11/948,903, entitled “Method And System For Conducting An Online Payment Transaction Using A Mobile Communication Device ” filed on Nov. 30, 2007, U.S. patent application Ser. No. 11/939,821, entitled “Method and System for Securing Transactions Made Through a Mobile Communication Device” filed Nov. 14, 2007, and U.S. patent application Ser. No. 11/933,351 entitled “Method and System for Purchasing Event Tickets Using a Mobile Communication Device”, filed Oct. 31, 2007, and U.S. patent application Ser. No. 11/933,321 entitled “Method and System for Adapting a Wireless Mobile Communication Device for Wireless Transactions,” filed Oct. 31, 2007 all of which are a continuation-in-part of U.S. patent application Ser. No. 11/467,441, entitled “Method and Apparatus for Completing a Transaction Using a Wireless Mobile Communication Channel and Another Communication Channel,” filed Aug. 25, 2006, which claims priority to U.S. Provisional Patent Application Nos. 60/766,171 and 60/766,172, both of which were filed December 31, 2005. All of the above-referenced patent applications are incorporated by reference herein in their entirety.

In another embodiment, a patient can transfer his/her medical records or other medical digital artifacts (medical ID, basic patient information, patient contact information, emergency contact information, primary care physician information, health insurance information including co-pay and deductibles, prescriptions, office visit summary, appointment cards, Electronic Medical Records (EMR), lab results, blood type, organ/donor status, vital signs, diagnostic data, immunization records, payments and transaction history, pictures, etc) from their NFC enabled mobile device to a remote device. For example, a patient may want to transfer their EMR to a new physician by simply waving their NFC enabled device near a physician's NFC enabled device. Alternatively, a patient may wish to transfer pictures from a sonogram to a personal computer or a social media page to share with family and friends. A patient can use NFC induction to transfer the digital medical records, conventional wireless communication including WIFI/GSM/CDMA, long range RF, or even inaudible sound waves. These methods are described in detail in U.S. patent application Ser. No. 13/215,069 entitled, “Transferring Data From An NFC Enabled Mobile Device to a Remote Device” filed on Aug. 22, 2011 and U.S. patent application Ser. No. 13/216,098 entitled “Streamlining NFC Transactions Using A Mobile Communication Device” filed on Aug. 23, 2011.

In another embodiment, NFC enabled devices can also be useful to patients who require daily or constant monitoring for insulin levels, asthma, high blood pressure, heart pace maker, etc. If the medical devices that monitor and capture these vital signs are equipped with an RFID or NFC transmitter, an NFC enabled mobile device where the data can be stored, charted, and even transmitted to their doctor and/or remote server where their EMR records are stored. To accomplish this, a patient, doctor, caregiver simply opens their mobile wallet, authenticates, and selects an option such as “Get diagnostic data from medical sensor”. Then the patient or health care professional holds the NFC device in proximity to the medical device or sensor and. During this process, the mobile wallet application will transfer a signal to the secure element to make the “Transfer diagnostic data secure element application which require authentication or SE_App6 or SE_App7 if no authentication is required” listed in FIG. 7 the active one or assign it priority of 1 so that the POP/POS/POE will select it first. If there are multiple secure elements, the secure element controller which maintains a directory of the location of secure elements and applications will direct the POP/POS/POE to the location of the active secure element application. If patient wishes to transfer this diagnostic data or digital artifacts to a physician, the process is outlined in Method 500. If they wish to transfer them to a remote device there are several processes identified in U.S. patent application Ser. No. 13/215,069, entitled “Transferring Data From An NFC Enabled Mobile Device to a Remote Device” filed on Aug. 22, 2011. A streamlined approach for capturing data is described in U.S. patent application Ser. No. 13/216,098 entitled “Streamlining NFC Transactions Using A Mobile Communication Device” filed on Aug. 23, 2011. This way patient's, don't have to write down their results and can have all of the data stored electronically.

To better control their diet which may be especially important for chronic health problems, patients may desire to track their food intake and can easily do so with their NFC enabled mobile device. The patient opens their mobile wallet and selects the option to get nutritional contents from products. During this process, the mobile wallet application will transfer a signal to the secure element to make the “Get nutritional values data secure element application which require authentication or SE_App11” listed in FIG. 7 the active one or assign it priority of 1 so that the POP/POS/POE will select it first. The Patient then simply tap or hold their NFC device near the labels on products that are equipped with RFID or NFC tags. Upon doing so, the key nutritional ingredients (e.g. carbohydrates, calories, fat, vitamins, and even potential allergies such a peanuts) can be transmitted to the NFC enabled mobile device. The mobile device can provide visual or audible alerts if the foods contain ingredients that are harmful to the patient (e.g. peanuts) or exceed a patient's maximum daily intake (e.g. carbohydrates). The patient can use the scanned data to create a meal plan and an electronic food diary and use it plan meals and to automatically track which food they consume. The electronic food diary can be transferred this to a health care professional who can use it to diagnosis the patient's condition and modify their diet and/or prescriptions accordingly.

To import the foods into a electronic food diary, the patient opens their mobile wallet and enters authentication information whereby they are authenticated by a remote server. Then, the patient selects an option such as “get food ingredients” from their mobile wallet. Afterwards, the patient taps or holds their NFC enabled mobile device next to the NFC/RFID label on the product. In doing so, the NFC enabled mobile device is able to “read” the NFC/RFID label on the product and transmit it to the secure element. The patient selects “Next” or a button with similar function on their mobile device and holds it near the next product. The patient continues this process until they have “scanned” all of the products using NFC and selects a button on their keypad or touch screen that represents “done” or “complete scanning”. The mobile wallet enables the patient to select which products to incorporate into a meal plan for a single meal, several meals, per day, per week, etc and calculates total calories, fat, carbohydrates etc. The mobile wallet also displays a chart comparing patients target diet levels which may be prescribed by a health care professional against the food selects for a given meal, day, week, etc. If the patient removes or adds a product to their meal plan, the mobile wallet will update the nutritional values automatically. The patient can transfer the chart to their personal computer using USB, IR, or even NFC induction if the PC, monitor, or keyboard are equipped with NFC. The patient can subsequently email it from the pc to their physician, caregiver, etc. They can also print it out.

NFC enabled devices can also be used to track patients in a hospital and serve as an effective tool for administering and monitoring applications. When a patient checks into a hospital, patients are given a with hospital ID bracelets as depicted in FIG. 4 that has an NFC or RFID chip embedded inside of the secure element affixed to the bracelet. The secure element has a unique ID for the patient which is stored in the management server. In the case where the bracelet contains just an RFID chip that is capable of being read only, the chip will have with the patient number encoded in it. When a health care professional visits the patient, they can use their NFC enabled mobile device equipped to scan the bracelet to confirm the patients ID using NFC induction. Upon scanning the bracelet and entering a PIN to confirm the identity of the nurse or doctor, the patient's id can be read from the bracelet and transmitted to a remote server using the second channel over conventional wireless communication WIFI or LAN. The patient's records are retrieved from the management server using the patient ID and displayed on the health care professional's NFC enabled mobile device. The doctor can confirm patients identify prior to applying medicine. The health care professional may have to enter their ID (or have their ID scanned) and enter authentication at the mobile hand held device such as a PIN, (biometrics such as a finger print scan, retina scan), or a combination of methods. Their authenticated against a management server.

In another scenario as described in method the hospital bracelet can contain a secure element with an NFC chip which is capable of both read and write capabilities. Similar to the scenario above, the secure element be encoded with a unique patient number. When a nurse or health care professional visits the patient, they can open their NFC enabled mobile device and select an option to “get the patient ID”. During this process, the mobile wallet application will transfer a signal to the secure element to make the “Get medical ID secure element application which require authentication or SE_App1” listed in FIG. 7 the active one or assign it priority of 1 so that the POP/POS/POE will select it first. If there are multiple secure elements, the secure element controller which maintains a directory of the location of secure elements and applications will direct the POP/POS/POE to the location of the active secure element application. Then, they hold their NFC enabled mobile device near the patient's hospital bracelet. In doing so, the health care professionals NFC enabled device causes induction, powers the secure element in the bracelet, and triggers an application in the patients secure element (i.e. Se_App1) and transfers the ID to the secure element of the health care professionals NFC enabled device. The health care professional can use the patients ID to retrieve the patient's electronic medial records from the management server and display them on their health care professional's NFC enabled mobile device. It is important to note to the health care professional may have to authenticate as described previously throughout this specification first prior to retrieving the patient's medical records

In addition to reading the patient's records, the health care professional can “write” electronically a prescribed medicine and dosage to the secure element of the bracelet. This is accomplished by using the mobile wallet on the NFC enabled mobile device to select the medicine and dosage. After this, the health care professional simply holds their NFC enabled device near the patient's bracelet. Upon doing so, the notes, prescription are transferred from the health care professionals secure element to the patients bracelet.

For critically ill patients who might be in Intensive Care Unit (ICU) or pre-natal car and have high sensitivity to dosage, NFC can be used to ensure that the right patients are getting the right medications and the correct dosages. Before a health care professional administers medicine n to a patient, they can first use their NFC enabled mobile device to scan the patients ID bracelet to confirm the identity of the patient and the prescribed dosage that might be stored in the patients bracelet or the patients ID can be used to retrieve the proper medication and dosage from the management server. Upon doing so, the prescribed medicine and dosage appears on the nurse/doctors NFC enabled mobile device. The health care professional can then select an option to verify the medical dosage from the medical dispenser. During this process, the mobile wallet application will transfer a signal to the secure element to make the “Get medical dosage secure element application which require authentication or SE_App12” listed in FIG. 7 the active one or assign it priority of 1 so that the POP/POS/POE will select it. If there are multiple secure elements, the secure element controller which maintains a directory of the location of secure elements and applications will direct the POP/POS/POE to the location of the active secure element application. The health care professional then hold their NFC enabled mobile device near the medicine dispenser which is equipped with an RFID or NFC tag. Upon doing so, the NFC mobile device reads the tag via induction and displays the contents of the medicine on the display of the NFC enabled mobile device. An application in the secure element (e.g. SE_App12) of the health care professionals NFC enabled mobile device and will determine if the prescribed medicine and dosage match the information identified on the medicine dispenser. If so, the secure element application will transmit this information to the mobile wallet where it is displayed for the health care professional to view along with a message for the health care professional to proceed with applying the medicine. If prescribed medicine and the medicine in the dispenser do not match, a warning (audible, visual, or both) is displayed on the health care professional NFC enabled mobile device followed by a message prompting the health care professional to verify the medicine/dosage is correct for the patient. If the health care professional is allowed to proceed with administering the medication, this information is recorded on the secure element the patient's bracelet along with date, time, location, nurse/doctor ID. The discrepancy between the prescribed medicine/dosage and the actual medicine/dosage is also transmitted to the remote server where it is also flagged with an urgent notification.

Since a patient has an RFID/NFC equipped bracelet, this lends itself also to surgery, if a patient may be going in for surgery on one of two different body parts (e.g. left leg vs right leg, left arm vs right arm, etc). Prior to the surgery, the patient is given a medical ID bracelet with not only a unique identifier, but also information which identifies the body part to be operated on. The bracelet can also be affixed to that body part if feasible. For example, if the patient is having surgery on their right knee, the bracelet might be applied to the right ankle in addition to one being applied to their wrist. This way, prior to surgery, the surgeon can scan the bracelet (s) with a NFC enabled mobile device and confirm that they are going to operate on the correct body part. As an additional layer of security, the surgeon can be required to confirm using the hand held mobile device which body part he/she plans to operate on after the scan and prior to commences. If pictures are required before, during or after the surgery, the physician can use the NFC enabled mobile device to take pictures and correlate them to the patient by tapping the NFC enabled mobile device near the patients hospital bracelet. After the patients ID comes up, the surgeon or someone else in the hospital staff takes picture, those pictures are automatically assigned to that patient.

In all of these scenarios, it is implied that medicines are prescribed a label. Today, most labels have barcodes which can be scanned. These can be utilized or they can be equipped with an RFID tag. When the medications are prepared, they can be entered into a computer which stores them in a remote server (aka “Cloud”) and subsequently a printer can print the information on an “RFID label” which is then affixed to the medicine in the same way a barcode label is applied. This will help with inventory tracking of medications and reduce any fraud or theft.

Although the present invention has been particularly described with reference to implementations discussed above, various changes, modifications and substitutes are can be made. Accordingly, it will be appreciated that in numerous instances some features of the invention can be employed without a corresponding use of other features. Further, variations can be made in the number and arrangement of components illustrated in the figures discussed above.

What is claimed is:

1. A method for transferring digital medical artifacts between NFC enabled devices, the method comprising:
receiving manual user authentication at the handheld mobile device based on a wherein the mobile device prompts the user for authentication, and further wherein authentication is performed by a remote server;
executing a health care secure element software application on a processor of a secure element for the transaction wherein the secure element coupled to a hand-held mobile device, wherein the secure element comprises:
a memory storing the health care secure element software application, user ID and DVC, wherein execution of the health care secure element software application generates the DVC and facilitates transfer of the ID and DVC to a point-of-presence terminal,
the processor of the secure element, wherein the processor is capable of near field communication, and further wherein the processor executes the active health care secure element software application with a priority of one stored in the memory, and
a wireless transceiver that sends transaction data, including the user ID and DVC associated with the executed health care secure element software application, through a second communication channel to the point-of-presence terminal, the second communication channel being different from a first communication channel through which the hand-held mobile device communicates voice and data, the payment application executed in response to a near field communication-based trigger from the point-of-presence terminal;
wirelessly receiving at the secure element transaction data from the point-of presence device
calculating a DVC at the secure element
wireless sending transaction data, including the user ID and DVC associated with the executed active secure element software application, through a second communication channel to the point-of-presence terminal,
wirelessly receiving a command to unlock the secure element; and
receiving digital artifacts at the secure element from the POP terminal which may be subsequently displayed on the mobile device.

2. The method of claim 1 where the point-of-presence device is a medical personnel NFC Enabled mobile device.

3. The method of claim 1 where the point-of-presence device is a kiosk

4. The method of claim 1 where the point-of-presence device is a medical sensor

5. The method of claim 1 where the point-of-presence device is a personal computer

6. The method of claim 1 where the secure element is unlocked by a remote server

7. The method of claim 1 where the secure element is unlocked by the mobile wallet application residing on the mobile device, wherein the mobile wallet decrypts a file downloaded by the remote server and retrieves the secure element key inside of the file.

8. The method of claim 1 where the medical digital artifacts are stored in another memory storage different than the memory storage the application resides in

9. The method of claim 1 where the different medical applications are stored in different secure elements, wherein the secure element controller manages the multiple secure element applications.

10. The method of claim 1 where information is loaded from a health care professional's NFC enabled device.

11. A method for using an NFC enabled hand held mobile communication device to receive digital medical artifacts from a health care professional's near field communication device.

12. The method of claim 1 where digital medical artifacts includes medical id, patient information, prescription, emergency contact information, physician information, vital signs, insurance information, etc

13. A method where an NFC controller manages multiple applications residing on secure element of a mobile device, the method consisting of
executing an NFC controller software application on a processor of a secure element for the transaction wherein the secure element coupled to a hand-held mobile device, wherein the secure element comprises
an NFC controller wherein the applications are controlled by an NFC controller
a memory storing the multiple secure element software application, user ID and DVC, wherein execution of the NFC controller application identifies the location of the active secure element software application and triggers the application, generates the DVC and facilitates transfer of the ID and DVC to a point-of-sale presence terminal,
the processor of the secure element, wherein the processor is capable of near field communication, and further wherein the processor executes the NFC controller application stored in the memory, and
a wireless transceiver that sends transaction data, including the user ID and DVC associated with the executed active secure element software application, through a second communication channel to the point-of-presence terminal, the second communication channel being different from a first communication channel through which the hand-held mobile device communicates voice and data, the active application executed in response to a near field communication-based trigger from the point-of-presence terminal.

14. A method for conducting an NFC payment transaction to pay for prescription and receiving digital artifacts after the transaction using a hand-held mobile device, the method comprising:
receiving manual user authentication at the handheld mobile device based on a wherein the mobile device prompts the user for authentication, and further wherein authentication is performed by a remote server;
executing a prescription application and a payment application on a processor of a secure element for the transaction wherein the secure element coupled to a hand-held mobile device, wherein the secure element comprises:
a memory storing the health care secure element applications including a prescription application, payment applications, and payment credentials, wherein execution of the prescription application facilitates transfer of a prescription and the payment application facilitates transfer of the payment credentials to a point-of-sale terminal,
the processor of the secure element, wherein the processor is capable of near field communication, and further wherein the processor executes the prescription application and payment application stored in the memory, and
a wireless transceiver that sends transaction data, including the prescription, co-pay, medical ID, co-pay, doctors digital signature, User ID, DVC, and payment credentials associated with the executed prescription and payment application, through a second communication channel to the point-of-sale terminal, the second communication channel being different from a first communication channel through which the hand-held mobile device communicates voice and data, the prescription application and payment application executed in response to a near field communication-based trigger from the point-of-sale terminal;
a radio/WIFI/Bluetooth wireless transceiver;
a microphone;
a speaker;
an audio cable'
an audio to digital converter;
a long range RF transceiver; and
a Secure Element Controller;
receiving manual user authentication at the point-of-sale device based on a determination by the point of sale terminal that authentication is required, wherein the point-of-sale terminal notifies a remote server which triggers the hand-held mobile device to prompt a user for authentication, and further wherein authentication is performed by a remote server that transmits notification of authentication to the point-of-sale terminal after payment credentials have been transferred to the point-of-sale terminal but prior to payment being processed and completed;
receiving manual user authentication at the mobile device to accept secondary digital medical artifacts
receiving a command to unlock the secure element; and
receiving secondary digital medical artifacts at the secure element from the POS terminal which are subsequently transferred to the mobile communication device where they are displayed.

15. A method for transferring medical digital artifacts between a point-of-presence device and a secure element that is external to a mobile communication device and not coupled to the NFC enabled mobile communication device, the method comprising:
executing an application on a processor of a secure element, the secure element including:
a memory storing the application and data, the execution of the application facilitating the transfer of the ID to the point-of-presence device,
a processor that executes the application stored in the memory,
a wireless transceiver that sends transaction data associated with the executed application through a first communication channel to the point-of-presence device, the first channel using a near field communication trigger;
a radio/WIFI/Bluetooth wireless transceiver;
a microphone;
a speaker;
an audio cable'
an audio to digital converter;
a long range RF transceiver;
a Secure Element Controller and
wirelessly transmitting transaction data from the secure element associated with the executed application through the first communication channel to the point-of-presence device using the wireless transceiver;
wirelessly receiving transaction data at the secure element associated with the executed application through the first near communication channel from the point-of-presence device using the wireless transceiver;
computing a dynamic verification code at the secure element based on the transaction data received from the point of presence device
wirelessly transmitting dynamic verification code to the point-of-presence device which subsequently transmits it through the second communication channel to the management server for authentication.
